SEQUENCE LISTING (14 sequences)

SEQ ID NO: 1 - DNA, 10 bp, Artificial Sequence
  Random MAP primer
aaatcggagc

SEQ ID NO: 2 - DNA, 10 bp, Artificial Sequence
  Random MAP primer
gtcntactcg

SEQ ID NO: 3 - DNA, 10 bp, Artificial Sequence
  Random MAP primer
tgcgcgatcg

SEQ ID NO: 4 - DNA, 10 bp, Artificial Sequence
  Random MAP primer
aacgtacgcg

SEQ ID NO: 5 - DNA, 10 bp, Artificial Sequence
  Random MAP primer
gcacgccgga

SEQ ID NO: 6 - DNA, 10 bp, Artificial Sequence
  Random MAP primer
cgggatccgc

SEQ ID NO: 7 - DNA, 10 bp, Artificial Sequence
  Random MAP primer
gcgaattccg

SEQ ID NO: 8 - DNA, 10 bp, Artificial Sequence
  Random MAP primer
ccctgcaggc

SEQ ID NO: 9 - DNA, 10 bp, Artificial Sequence
  Random MAP primer
ccaagcttgc

SEQ ID NO: 10 - DNA, 10 bp, Artificial Sequence
  Random MAP primer
aagatagcgg

SEQ ID NO: 11 - DNA, 10 bp, Artificial Sequence
  Random MAP primer
ggatctgaac

SEQ ID NO: 12 - DNA, 10 bp, Artificial Sequence
  Random MAP primer
ttgtctcagg

SEQ ID NO: 13 - DNA, 10 bp, Artificial Sequence
  Random MAP primer
catcccgaac

SEQ ID NO: 14 - DNA, 10 bp, Artificial Sequence
  Random MAP primer
ggactccacg

(19) United States
(12) Plant Patent Application Publication
(10) Pub. No.: US 2004/0194175 P1
(43) Pub. Date: Sep. 30, 2004

(54) Citral rich high yielding lemongrass plant 'Nima' of Cymbopogon flexuosus

(76) Inventors: Raj Kishori Lal, Uttar Pradesh (IN); Hari Om Misra, Uttar Pradesh (IN); Jawahar Ram Sharma, Uttar Pradesh (IN); Nilakshi Singh, Uttar Pradesh (IN); Ajit Kumar Shasany, Uttar Pradesh (IN); Ali Arif Naqvi, Uttar Pradesh (IN); Janak Raj Bahl, Uttar Pradesh (IN); Arun Prasad, Uttar Pradesh (IN); Suman Preet Singh Khanuja, Uttar Pradesh (IN)

Correspondence Address: BIRCH STEWART KOLASCH & BIRCH, PO BOX 747, FALLS CHURCH, VA, 22040-0747, US

(21) Appl. No.: 10/401,753

(22) Filed: Mar. 31, 2003

(51) Int. Cl. A01H 5/00

(52) U.S. Cl. PLT/258

(57) ABSTRACT

The present invention relates to a new and distinct high essential oil variety of lemon grass (Cymbopogon flexuosus) named as ‘Nima’. Further the invention relates to the high content of the monoterpene citral in the essential oil. This novel variety ‘Nima’ of the lemon grass is a selection from the open pollinated seedlings of the seeds obtained from another Cymbopogon flexuosus variety OD 19 from the germplasm collection of CIMAP. This variety is propagated through vegetative means using the slips and stable for commercial cultivation.

### FIELD OF THE PRESENT INVENTION

The present invention relates to a new and distinct high essential oil variety of lemon grass (Cymbopogon flexuosus) named as ‘Nima’. Further the invention relates to the high content of the monoterpene citral in the essential oil. This novel variety ‘Nima’ of the lemon grass is a selection from the open pollinated seedlings of the seeds obtained from another Cymbopogon flexuosus variety OD 19 from the germplasm collection of CIMAP. This variety is propagated through vegetative means using the slips and stable for commercial cultivation.

### BACKGROUND AND PRIOR ART REFERENCES OF THE INVENTION

Lemongrass (Cymbopogon flexuosus), family—Poaceae is an aromatic grass species and is commonly known as ‘East Indian Lemongrass’. It is a vegetatively (also seed) propagated perennial, multicut crops in the tropics. The prefix ‘Lemon’ owes to its typical lemon—like odour, released from leaves on maceration, which is mainly due to the presence of citral as a cyclic monoterpene. The lemongrass oil as such is widely used in perfumery, soaps and cosmetics to obtain typical lemon note. Beside, it's an important source of citral, which is used in perfumery and medicine. While citral forms a significant raw material for confectionery and beverages, it is the principal source of E-ionone which is extensively used for the synthesis of vitamin A and a number of a chemicals including synthetic violet perfumes. Due to limited improved varieties for high oil yield and citral content its cultivation is not popular among farmers. Therefore, there is a need to develop superior varieties of lemongrass for high oil yield and citral content.

### OBJECT OF THE PRESENT INVENTION

The main object of the invention is, therefore, to develop a new and distinct high oil rich in citral contents variety of lemon grass (Cymbopogon flexuosus) which can produce high amount of lemongrass oil with high citral content.

### SUMMARY OF THE PRESENT INVENTION

The present invention relates to a new and distinct high essential oil variety of lemon grass (Cymbopogon flexuosus) named as ‘Nima’. Further the invention relates to the high content of the monoterpene citral in the essential oil. This novel variety ‘Nima’ of the lemon grass is a selection from the open pollinated seedlings of the seeds obtained from another Cymbopogon flexuosus variety OD 19 from the germplasm collection of CIMAP. This variety is propagated through vegetative means using the slips and stable for commercial cultivation.

### DETAILED DESCRIPTION OF THE PRESENT INVENTION

Accordingly, the present invention provides a new and distinct variety of Cymbopogon flexuosus ‘Nima’ producing high essential oil, rich in citral and the variety is characterized by the morphological/agronomic features as described in Table 1.

TABLE 1
Morphological/agronomic feature of var. Nima

| | Elite clone/strain | Checks | | |
|---|---|---|---|---|
| Attributes | CIMAP/S 49 (NIMA) | PRAGATI | PRAMAN | CKP 25 |
| Plant height | 100.00 | 145.00 | 125.00 | 160.00 |
| (cm) | | | | |
| Growth habit | Closed/erect | open | semi | semi |
| | | | closed | open |
| No. of tillers/ | 65 | 45 | 50 | 55 |
| plant | | | | |
| Herb yield/ | 289 | 215 | 240 | 185 |
| year (q/ha) | | | | |
| Oil content (%) | 0.86 | 0.57 | 0.56 | 0.93 |
| Oil yield | 248 | 122 | 124 | 172 |
| (kg/ha) | | | | |
| Citral (%) | 89.06 | 82.04 | 82.23 | 79.07 |

The strain CIMAP/S-49 (now on wards will be referred as ‘Nima’) has medium tall height, with small, dark purple tinged stem, high number of tillers/plant and dark green broad leaves as the distinct morphological features (colour categories are described later).

Relative performance of ‘Nima’ in different yield evaluation trials is presented in the following Table 2:

TABLE 2
Mean performance (⁻X) of elite clones in
different yield trials of lemongrass

S.No. Entries Herb yield Oil content (%) Oil yield Citral content (%)
IET BST PST IET BST PST IET BST PST IET BST PST
(g/plot) (kg/plot) (ql/ha/year) (g/plot) (g/plot) (kg/ha/year)
Plot size sqm = 0.50 3.75 100 0.50 3.75 100 0.50 3.75 100 0.50 3.75 100
RBD. Reps. = 3 3 −3 3 −3 3 −3 3
Years = 1 2 2 1 2 2 1 2 2 1 2 2
Cuttings = 3 6 6 3 6 6 3 6 6 3 6 6

1. CIMAP/S 49 0.56 9.32 289 0.79 0.89 0.86 4.13 82.32 248 89.58 90.32 89.06

2. CIMAP/K 10.47 6.92 -- 0.32 0.53 -- 1.51 37.46 -- 60.17 81.31--

3. CIMAP/A 4 0.81 -- -- 0.46 -- -- 3.73 -- -- 2.64-- --

4. CIMAP/S 21 0.26 6.67 -- 0.44 0.39 --1.22 25.89-- 82.53 71.68 --

5. CIMAP/S 1 0.31 7.22 -- 0.62 0.55 -- 1.94 56.71-- 83.23 89.24--

6. CIMAP/S 40.29 8.01 -- 0.70 0.68 -- 2.07 53.56-- 85.23 85.29--

7. Pragati 0.30 6.11 215 0.47 0.62 0.57 1.39 34.32 122 84.03 82.70 82.04

8. Praman 0.43 6.58 240 0.50 0.63 0.56 2.10 39.59 124 79.97 77.75 82.23

9. OD 19 0.29 5.40 -- 0.52 0.56 -- 1.45 29.15 --83.50 84.72--

10. Krishana 0.24 6.48 -- 0.93 0.94 -- 2.18 56.66 - - 80.53 79.53 --

11. Cauvery 0.24 4.67 -- 0.65 0.70 -- 1.55 27.00 --81.17 79.36 --

12. CKP 25 0.34 6.87 185 0.87 0.98 0.93 3.25 70.29 172 77.27 77.65 79.07

LSD (5%) 0.17 1.54 -- 0.07 0.25 -- 10.56 10.30 -- 1.27 7.42 --

(1%) 0.232.06 -- 0.18 0.18 -- 14.35 13.09 -- 1.73 9.99--

### BRIEF DESCRIPTION OF THE ACCOMPANYING DRAWINGS

FIG. 1 shows the plant “Nima” in the field.

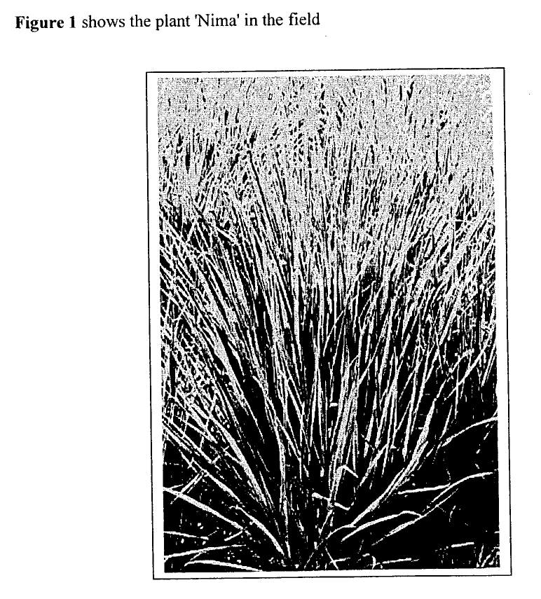

FIG. 2 shows unique RAPD profile of “Nima”.

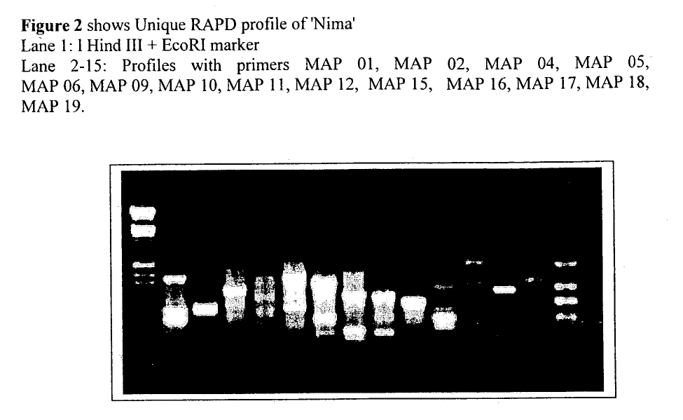

### TAXONOMIC DESCRIPTION OF THE PLANT “NIMA”

The aromatic perennial grass ‘Nima’ (family-Poaceae) attains a height up to 100 cm. The morphological description has been provided below:

1. Genus: Cymbopogon.

2. Species: flexeosus L.

3. Family: Poaceae.

4. Common name: Lemon grass.

5. Plant height: 100.00 cm.

6. Growth habit: Erect and bushy.

7. Stem: Culm, Moderately bold Rudimentary, leaves coming out in whorls.

Thickness.—1.0 to 2.0 cm.

Number of tillers per plant.—55 to 70.

Number of leaves per tiller.—6 to 15.

Colour.—Yellow Green Group (145 D).

8. Leaf: Seathed, sessile.

Colour of upper surface.—Green Group (137B).

Colour of lower surface.—Green Group (137A).

Texture.—Rough.

Surface.—Glabrous.

Shape.—Linear.

Margin.—Undulating.

Tip.—Acuminate.

Base.—Attenuate.

Leaf sheath.—Purple group (79A) upper surface, Grayed green (139A) lower surface with purple streaks (79A).

Area.—82.55 sq cm.

Length.—40 to 80 cm leaf lamina, 15 to 25 cm leaf sheath.

Width.—1.5 to 2.5 cm leaf lamina, 1.0 to 1.5 cm leaf sheath.

9. Inflorescence: Panicle.

Florets.—Purple violet (80C), Glumes 3.0 to 3.5 mm, Awns 11.0 to 14.0 mm long, lowest pedicle of sessile spikelet is slightly swollen and connate to the base of rachis.

Arrangement.—Racemose.

Time to flowering.—February.

Lastingness of bloom.—February to April.

Seeds.—Purple violet (80C).

10. Oil content in the fresh herb (%): 0.8 to 0.9.

11. Oil quality:

Citral.—85.0 to 90.0%.

Limonene.—0.1 to 0.5%.

Linalool.—0.1 to 0.5%.

Geraniol.—1.0 to 2.0%.

Geranyl acetate.—0.5 to 1.5%.

12. Herbage (Shoot biomass): 150 to 300 quintal/ha/year.

The colour codes are according to the RHS colour chart published by The Royal Horticultural Society, 80 Vincent Square, London SWIP 2PE, 1995.

Breeding History of ‘Nima’

The vegetative slips of Cymbopogon flexuosus ‘OD-19’ (a released variety of lemongrass from Lemon Grass Research Center, Odakalli, Kerala, India) were obtained from the National Gene Bank of Medicinal and Aromatic plants housed at Central Institute of Medicinal and Aromatic Plants, Lucknow. These were grown in the field of CIMAP in the year 1994-1995 and the seedlings obtained from the seeds of ‘O.D. 19’ were primarily screened for high herb and oil yield with high citral content in the year 1995-1996. The slips were planted in the month of July and harvested 3 times in the year in 4 months interval. Fresh leaves were collected in the during harvesting from the field grown plants and oil was extracted from 100 g plant samples in 3 replicates by hydro-distillation using Clevenger's apparatus. Oil content in terms of oil percentage was calculated as the mean of 3 samples. GLC analysis for major oil constituents was performed on Varian CX-3400 using a 30 m×0.25 mm (0.25 m) Supelcowax-10 column. The injector and detector temperature were maintained at 200 and 225° C. respectively, with oven temperature programmed from 60 to 200 ° C. at the rate of 7° C. min⁻¹ increase, with initial and final holds of 2 and 5 minutes respectively. Hydrogen gas was used as carrier at the rate of 1 ml min⁻¹ and 0.1 ml of sample was injected with a split ratio of 1:50. Data were processed in the electronic integrator Varian 4400 and the identification was based on retention time of spiked authentic samples of different compounds and retention indices calculations. As a result, six superior plants (CIMAP/S 49, CIMAP/K1, CIMAP/A4, CIMAP/S1, CIMAP/S21 and CIMAP/S40) were identified for above parameters.

They were then evaluated in Initial Evaluation Trial (IET) during 1996-1997 along with six checks (released varieties) ‘OD 19’,‘Pragati’, ‘Praman’, ‘Krishna’, ‘Cauvery’ and ‘CKP 25’, where the five clones, namely CIMAP/S 49, CIMAP/KI, CIMAP/S1, CIMAP/S21 and CIMAP/S40 were found to be highly promising for high herb, oil yield and better quality of essential oil. Then, these superior selected clones were evaluated in Bench Scale Trial (BST) for two years where these were compared to all the checks during 1997-1998 and 1998-1999. The high herb and oil yielding clone CIMAP/S 49 with high citral content was then placed in Pilot Scale Trials (PST) for two years along with three best checks varieties (‘Pragati’, ‘Praman’, ‘and ‘CKP 25‘) during 1998-1999 and 1999-2000. This elite clone /strain CIMAP/S 49 maintained its superiority over all the checks for herb, oil yield and citral content. The elite strain was named as variety ‘Nima’.

Yield trials were conducted at CIMAP's farm, Lucknow (UP, India) and herbage, oil yield and citral content were observed to be higher (as in Tables 2) in the new genotype ‘Nima’, in comparison to other existing varieties. Similarly, no variants of any kind has been observed since 1997-2000 and till the date of filing the patent, indicating the stability and uniformity of the genotype. Further, the comparative herbage and oil yields of ‘Nima’ were significantly higher in comparison to other varieties/genotypes in different years and seasons. The plant can be multiplied vegetatively for propagation through slips for commercial cultivation and can be grown in monoculture and other crop may follow after harrowing the field with the disk harrow.

Accordingly the invention provides a new and distinct citral rich of lemongrass which can be stably propagated through vegetative means and having the following combinations of characters established as distinctiveness from the known varieties of Cymbopogon flexuosus,

a. Greenish upper surface (137B), greenish lower surface (137A) Purple violet (80C) florets, and rudimentary culms supporting the leaves in whorls,

b. randomly amplified polymorphic DNA(RAPD) profile for polymerised chain reaction(PCR)amplified DNA segment using 14 primers distinct from the other existing varieties,

c. highest biomass (250 to 300 q/ha/year) and highest oil yield (220 to 260 kg/ha/year) in comparison to other available checks.

d. highest citral content (85 to 90%) in comparison to other genotypes.

e. essential oil with the combination of moterpenes like Citral: 85.0 to 90.0%, Limonene: 0.1 to 0.5%, Linalool: 0.1 to 0.5%, Geraniol: 1.0 to 2.0%, Geranyl acetate: 0.5 to 1.5% in addition to other unidentified monoterpene fractions totaling to 100%.

The genotype ‘Nima’ possessing green (137B) colour upper surface, and green (137A) lower surface (Photograph#1), purple (80C) colour florets produces highest biomass (250 to 300 q/ha/year) and highest oil yield (220 to 260 kg/ha/year) in comparison to other available checks. The citral content of the new genotype is higher (85 to 90%) in comparison to other genotypes.

RAPD Analysis

The genotype Nima is distinct in its RAPD profile in comparison to ‘Pragati’, ‘Praman’, ‘CKP25’ and ‘Krishana’. Though the genotype ‘Nima’ was a seed progeny of the released variety ‘OD 19’, still the RAPD pattern was different with the parent, which indicates gross genome rearrangements in the new genotype.

The random primers used for developing the RAPD patterns are as follows:

Primers Base sequence

| | |
|---|---|
| MAP 01 | 5′ AAA TCG GAG C 3′ |
| MAP 02 | 5′ GTC XTA CTC G 3′ |
| MAP 04 | 5′ TGC GCG ATC G 3′ |
| MAP 05 | 5′ AAC GTA CGC G 3′ |
| MAP 06 | 5′ GCA CGC CGG A 3′ |
| MAP 09 | 5′ CGG GAT CCG C 3′ |
| MAP 10 | 5′ GCG AAT TCC G 3′ |
| MAP 11 | 5′ CCC TGC AGG C 3′ |
| MAP 12 | 5′ CCA AGC TTG C 3′ |
| MAP 15 | 5′ AAG ATA GCG G 3′ |
| MAP 16 | 5′ GGA TCT GAA C 3′ |
| MAP 17 | 5′ TTG TCT CAG G 3′ |
| MAP 18 | 5′ CAT CCC GAA C 3′ |
| MAP 19 | 5′ GGA CTC CAC G 3′ |

(MAP—Medicinal and Aromatic Plants)

Using the above primers, the distinct RAPD profile of the new genotype ‘Nima’ was deduced (Photograph # 2 ).

The genotype ‘Nima’ or the variety ‘Nima’ was developed at Central Institute of Medicinal and Aromatic Plants (CIMAP), Lucknow a constituent of the Council of Scientific and Industrial Research (CSIR), India under the genetic improvement programme of Lemongrass. The complete RAPD profile for PCR amplified DNA using 14 primers (as mentioned earlier) is available providing evidence of distinctiveness of the genotype ‘Nima’.

After ascertaining the distinctiveness of the genotype ‘Nima’ from the progenitor ‘OD 19’ through RAPD profiling the analysis was narrowed down to ‘Pragati’, ‘Praman’, ‘CKP25’ and ‘Krishana’ for comparison. Genotypic pair analysis was used for assessing the genetic/molecular distances. The following table gives the extent of similarity among these three genotypes in paired test.

TABLE 3
Similarity indices (Percentage/100) for commonality of amplified
bands (paired analysis)

| | Pragati | Praman | CKP 25 | Nima | Krishna |
|---|---|---|---|---|---|
| Pragati | 1.000 | | | | |
| Praman | 0.481 | 1.000 | | | |
| CKP 25 | 0.423 | 0.602 | 1.000 | | |
| Nima | 0.674 | 0.462 | 0.524 | 1.000 | |
| Krishna | 0.724 | 0.484 | 0.518 | 0.785 | 1.000 |

From this analysis the genotype ‘Nima’ was found to be 21.5% to 53.8% diverse compared to the check varieties and hence completely new and distinct.

1. A new and distinct citral rich plant of lemongrass (Cymbopogon flexuosus) which can be stably propagated through vegetative means and having the following combinations of characters established as distinctiveness from the known varieties;
a. Greenish upper surface (137B), greenish lower surface (137A) Purple violet (80C) florets, and rudimentary culms supporting the leaves in whorls,
b. randomly amplified polymorphic DNA(RAPD) profile for polymerised chain reaction(PCR)amplified DNA segment using 14 primers distinct from the other existing varieties,
c. highest biomass (250 to 300 q/ha/year) and highest oil yield (220 to 260 kg/ha/year) in comparison to other available checks.
d. highest citral content (85 to 90%) in comparison to other genotypes.
e. essential oil with the combination of moterpenes like Citral: 85.0 to 90.0%, Limonene: 0.1 to 0.5%, Linalool: 0.1 to 0.5%, Geraniol: 1.0 to 2.0%, Geranyl acetate: 0.5 to 1.5% in addition to other unidentified monoterpene fractions totaling to 100%.
